(19) United States
(12) Patent Application Publication
(10) Pub. No.: US 2023/0274575 A1
(43) Pub. Date: Aug. 31, 2023

(54) BLOOD VESSEL DETERMINATION DEVICE, BLOOD VESSEL DETERMINATION METHOD AND NON-TRANSITORY STORAGE MEDIUM

(71) Applicant: LAPIS SEMICONDUCTOR CO., LTD., KANAGAWA (JP)

(72) Inventor: KAZUNORI FUJIWARA, KANAGAWA (JP)

(21) Appl. No.: 18/113,159

(22) Filed: Feb. 23, 2023

(30) Foreign Application Priority Data
Feb. 25, 2022 (JP) 2022-028510

(51) Int. Cl. G06V 40/14; G06V 10/60; G06V 10/145

(52) U.S. Cl. CPC G06V 40/14; G06V 10/60; G06V 10/145; G06V 2201/03

(57) ABSTRACT

A blood vessel judgment device acquires an image captured while near-infrared light is illuminated at a part of a body. Based on brightness values of a plurality of pixels configuring the acquired image, the blood vessel determination device determines that a predetermined number of pixels counted in order of brightness value from a pixel with a lowest brightness value represent blood vessels.

### CROSS-REFERENCE TO RELATED APPLICATION

This application is based on and claims priority under 35 USC 119 from Japanese Patent Application No. 2022-028510 filed on Feb. 25, 2022, the disclosure of which is incorporated by reference herein.

### BACKGROUND

### Technical Field

The present disclosure relates to a blood vessel determination device, a blood vessel determination method and a non-transitory storage medium memorizing a blood vessel determination program.

### Related Art

A device that enables non-invasive observation of blood vessels in the palm of a hand or the like is known (for example, see Japanese Patent Application Laid-Open (JP-A) No. 2017-209315).

From a first image set formed of plural images of a region containing blood vessels of a subject, a processing apparatus disclosed in JP-A No. 2017-209315 identifies, from changes in brightness values of portions of the images, a changing range in which changes in blood flow in the blood vessels are large and states of change of the brightness values satisfy a predetermined condition. The processing apparatus generates a second image set by selecting plural images included in the changing range, and performs computation processing on signals of plural images including the images of the second image set.

When positions of blood vessels are extracted from images based on plural images as in the processing apparatus disclosed in JP-A No. 2017-209315, sophisticated image processing must be conducted on the images. When a sophisticated technique is used, the processing load of a microcomputer is great. Therefore, when a technique such as that disclosed in JP-A No. 2017-209315 is used for extracting blood vessels from images, a computer capable of dealing with this processing load must be provided, and costs for blood vessel determination processing are high.

The present disclosure is made in order to solve the problem described above. An object of the present disclosure is to provide a blood vessel determination device, a blood vessel determination method and a memory medium memorizing a blood vessel determination program that may determine blood vessels appearing in an image easily.

### SUMMARY

A blood vessel determination device relating to the present disclosure includes: an acquisition section that acquires an image captured while near-infrared light is illuminated at a part of a body; and a determination section that, based on brightness values of plural pixels configuring the image acquired by the acquisition section, determines that a predetermined number of pixels counted in order of brightness value from a pixel with a lowest brightness value represent blood vessels.

A blood vessel determination method relating to the present disclosure includes a computer executing processing including: acquiring an image captured while near-infrared light is illuminated at a part of a body; and, based on brightness values of a plurality of pixels configuring the acquired image, determining that a predetermined number of pixels counted in order of brightness value from a pixel with a lowest brightness value represent blood vessels.

A non-transitory storage medium memorizing a blood vessel determination program relating to the present disclosure memorizes a blood vessel determination program for causing a computer to execute processing including: acquiring an image captured while near-infrared light is illuminated at a part of a body; and, based on brightness values of a plurality of pixels configuring the acquired image, determining that a predetermined number of pixels counted in order of brightness value from a pixel with a lowest brightness value represent blood vessels.

According to the present disclosure, a blood vessel appearing in an image may be determined easily.

### BRIEF DESCRIPTION OF DRAWINGS

FIG. 1 is a diagram showing an example of structures of a blood vessel determination system according to an exemplary embodiment.

FIG. 2 is a functional block diagram showing an example of a configuration of the blood vessel determination device according to the exemplary embodiment.

FIG. 3 is a diagram showing an example of a histogram depicting a distribution of brightness values in an image of a finger.

FIG. 4 is a diagram for describing a state in which light outputted from a lamp impinges on an image without being transmitted through a finger.

FIG. 5 is a diagram showing an example of image processing results.

FIG. 6 is a diagram showing another example of image processing results.

FIG. 7 is a diagram showing a structural example of a computer of the blood vessel determination device according to the exemplary embodiment.

FIG. 8 is a flowchart showing an example of processing that is carried out by the blood vessel determination device according to the exemplary embodiment.

### DETAILED DESCRIPTION

Below, an exemplary embodiment relating to the present disclosure is described in detail with reference to the drawings.

### Exemplary Embodiment

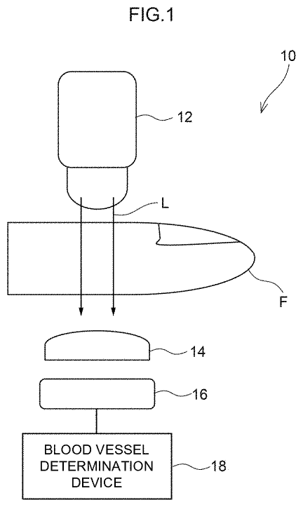

FIG. 1 is a diagram showing an example of structures of a blood vessel determination system 10 according to the exemplary embodiment. As shown in FIG. 1, the blood vessel determination system 10 is provided with a lamp 12, a lens 14, a camera 16 and a blood vessel determination device 18. As is shown in FIG. 1, a finger F, which is an example of a part of a body, is placed between the lamp 12 and the lens 14. Near-infrared light L that is outputted from the lamp 12 is illuminated onto the finger F. The near infrared light L transmits through the finger F. The lamp 12 is an LED light that outputs near-infrared light in the vicinity of 800 nm to 1000 nm. A mode is possible in which the near-infrared light outputted from the lamp 12 is not transmitted through the finger F but reflected at the finger F so as to acquire an image of the finger F. The camera 16 captures images of the finger F.

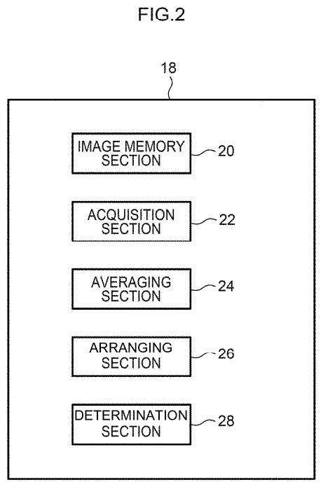

As shown in FIG. 2, the blood vessel determination device 18 is equipped in functional terms with an image memory section 20, an acquisition section 22, an averaging section 24, a arranging section 26 and a determination section 28. The blood vessel determination device 18 determines blood veins that appear in the images acquired by the camera 16.

Images of the finger F captured by the camera 16 while the near-infrared light is illuminated are stored at the image memory section 20. Plural images that are captured in a predetermined time interval are stored at the image memory section 20.

The acquisition section 22 acquires images of the finger F by reading the images from the image memory section 20. The acquisition section 22 acquires images of respective times that are stored at the image memory section 20.

By averaging brightness values of pixels at corresponding positions contained in a plural number of images acquired by the acquisition section 22, the averaging section 24 generates an averaged image in which the plural images are averaged. Thus, irregularities such as pulse waves and the like that appear in the images of the finger F may be moderated.

The arranging section 26 arranges brightness values of plural pixels configuring the averaged image generated by the averaging section 24 into ascending order.

Based on the brightness values of the plural pixels that have been arranged by the arranging section 26, the determination section 28 determines that, of the pixels configuring the averaged image, a first predetermined number of pixels counted in order of brightness value from a pixel with a lowest brightness value represent blood vessels, and determines that a second predetermined number of pixels counted in order of brightness value from a pixel with a highest brightness value do not represent blood vessels.

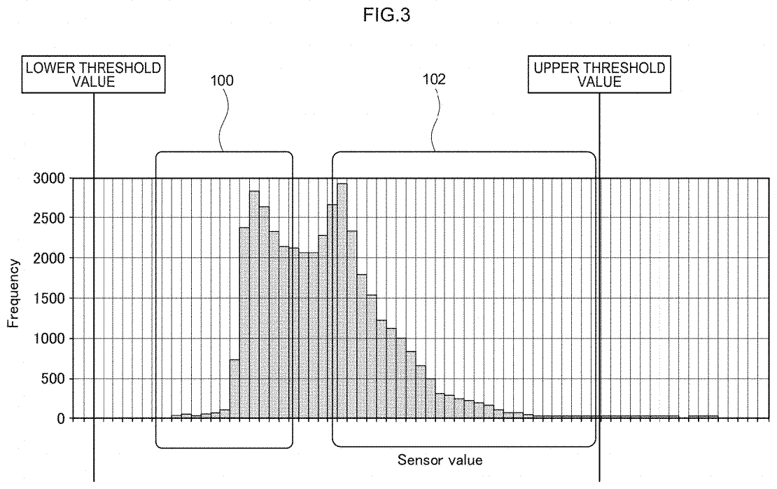

FIG. 3 shows a diagram for describing a method of determining blood veins according to the present exemplary embodiment. The graph shown in FIG. 3 is a histogram in which the horizontal axis represents brightness values of pixels contained in an image and the vertical axis represents frequencies of occurrence of these brightness values. The example in FIG. 3 is a histogram depicting a brightness value distribution for an image of a finger that has 40,000 pixels. As shown in FIG. 3, two peaks are formed in the brightness value distribution of this finger image. It is surmised that in the brightness value distribution with these two peaks, the peak with the higher brightness value represents regions that are not blood vessels and the peak with the lower brightness value represents regions of blood vessels.

Accordingly, the determination section 28 according to the present exemplary embodiment determines that, of the plural pixels configuring the images acquired by the acquisition section 22, the first predetermined number of pixels counted from a pixel with a lowest brightness value represent blood vessels and the second predetermined number of pixels counted from a pixel with a highest brightness value do not represent blood vessels. For example, in relation to an image with 40,000 pixels, the first predetermined number used for determining what is blood vessels can be set to 10,000 and the second predetermined number used for determining what is not blood vessels can be set to 10,000. The first predetermined number and the second predetermined number are specified in advance.

As is also shown in FIG. 3, in the present exemplary embodiment a lower threshold value, which is an example of a first threshold value, and an upper threshold value, which is an example of a second threshold value, are specified. These threshold values are specified in advance.

Thus, more specifically, the determination section 28 determines that, among pixels with brightness values higher than the lower threshold value, the first predetermined number of pixels counted from a pixel with a lowest brightness value represent blood vessels. In addition, the determination section 28 determines that, among pixels with brightness values smaller than the upper threshold value, the second predetermined number of pixels counted from a pixel with a highest brightness value do not represent blood vessels. Therefore, because pixels with very small brightness values and pixels with very high brightness values are excluded from determination, blood vessels appearing in the images may be determined easily and accurately.

In the example shown in FIG. 3, as an example, pixels falling within the region labeled 100, which corresponds to an aggregation of the first predetermined number of pixels with brightness values higher than the lower threshold value, are determined to be of blood vessels. Meanwhile, pixels falling within the region labeled 102, which corresponds to an aggregation of the second predetermined number of pixels with brightness values lower than the upper threshold value, counted from pixels with higher brightness values, are determined to not be blood vessels.

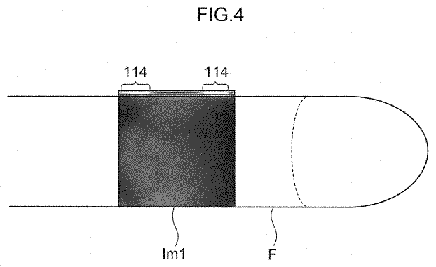

FIG. 4 shows an example in which the lamp 12 itself impinges on an image. The example in FIG. 4 is an example of a conceptual view depicting a state in which the lamp 12 is viewed from the camera 16. As shown in FIG. 4, when the lamp 12 itself impinges in an image, pixels in regions of the lamp 12 have high brightness values. Image Im1 shown in FIG. 4 has regions 114 in which the lamp 12 itself impinges. These regions are regions that cannot be identified as being blood vessels or not blood vessels. Accordingly, these regions are excluded by the upper threshold value. More specifically, the regions 114 are aggregations of pixels with brightness values higher than the upper threshold value, and therefore may be determined by the determination section 28 as being regions that cannot be said to be blood vessels or to not be blood vessels.

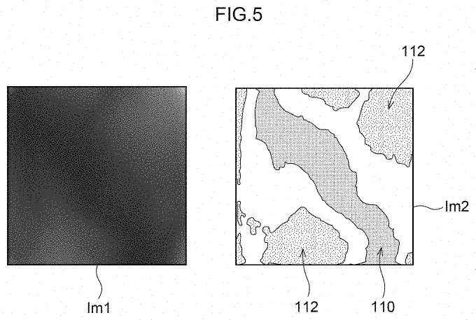

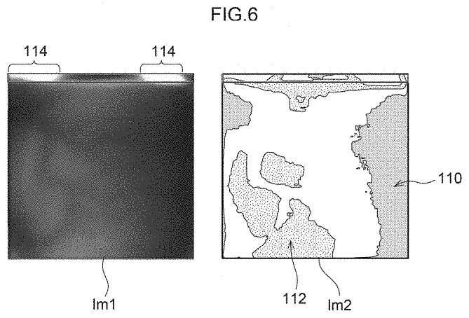

FIG. 5 and FIG. 6 show examples in which blood vessels appearing in images are determined by the technique of the present exemplary embodiment. In the examples shown in FIG. 5 and FIG. 6, the images Im1 are original images and the images Im2 are processed images. In the examples shown in FIG. 5 and FIG. 6, general regions of blood vessels and regions that are not blood vessels are extracted as intended.

In FIG. 5 and FIG. 6, as shown in the processed images Im2, regions marked 110 are determined to be blood vessels and regions marked 112 are determined not to be blood vessels. The regions 114 in which light from the lamp 12 impinges are present in FIG. 6. However, these regions correspond to white regions in the processed image Im2 and are determined to be regions that cannot be said to be blood vessels or to not be blood vessels.

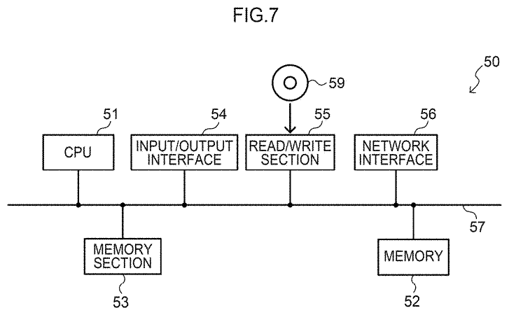

The blood vessel determination device 18 may be realized by, for example, a computer 50 as shown in FIG. 7. The computer 50 may be what is known as a microcomputer. The computer 50 is provided with a central processing unit (CPU) 51, a memory 52 that serves as a temporary memory region, and a nonvolatile memory section 53. The computer 50 is further provided with an input/output interface 54 to which input/output devices and the like (not shown in the drawings) are connected, and a read/write section 55 that controls reading and writing of data at a recording medium 59. The computer 50 is also provided with a network interface 56 that is connected to a network such as the Internet or the like. The CPU 51, memory 52, memory section 53, input/output interface 54, read/write section 55 and network interface 56 are connected to one another via a bus 57.

The memory section 53 may be realized by a hard disk drive (HDD), solid-state drive (SSD), flash memory or the like. A program for causing functioning of the computer 50 is memorized at the memory section 53, which serves as a memory medium. The CPU 51 reads the program from the memory section 53, loads the program into the memory 52, and sequentially executes processes of the program.

Now, operation of the blood vessel determination system 10 according to the exemplary embodiment is described.

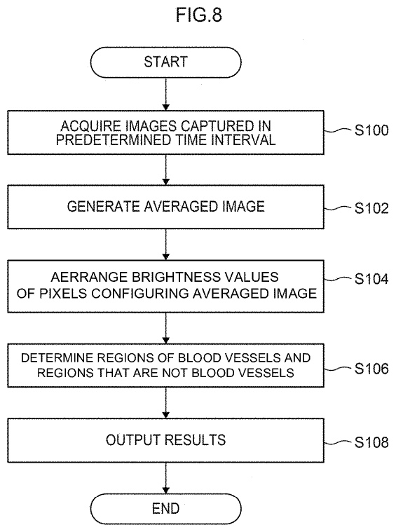

When near-infrared light L is outputted from the lamp 12 and images of a finger F are captured by the camera 16, the blood vessel determination device 18 acquires the images and successively stores the images at the image memory section 20. Hence, when the blood vessel determination device 18 receives command signals for determining blood vessels, the blood vessel determination device 18 executes the processing routine shown in FIG. 8.

In step S100, the acquisition section 22 acquires images stored at the image memory section 20 from respective times in a predetermined time interval.

In step S102, the averaging section 24 averages brightness values of pixels at corresponding positions contained in the plural images acquired in step S100, thus generating an averaged image in which the plural images are averaged.

In step S104, the arranging section 26 arrangs the brightness values of the plural pixels configuring the averaged image generated in step S102 into ascending order.

In step S106, based on the brightness values of the plural pixels arranged in step S104, the determination section 28 determines that, of the pixels configuring the averaged image, the first predetermined number of pixels counted in order of brightness value from pixels with small brightness values that have brightness values higher than the lower threshold value are pixels of blood vessels. In addition, the determination section 28 determines that, of the pixels configuring the averaged image, the second predetermined number of pixels counted in order of brightness value from pixels with high brightness values that have brightness values lower than the upper threshold value are pixels that are not of blood vessels.

In step S108, based on the determination results obtained in step S106, the determination section 28 generates a processed image Im2, as in the examples shown in FIG. 5 and FIG. 6, and outputs this processed image Im2 to serve as results.

As described above, the blood vessel determination device according to the exemplary embodiment acquires an image captured while near-infrared light is illuminated at a finger, and based on brightness values of plural pixels configuring the image, the blood vessel determination device determines that a predetermined number of pixels counted in order of brightness value from pixels with small brightness values are of blood vessels. More specifically, the blood vessel determination device 18 determines that, among the respective plural pixels configuring the image, the first predetermined number of pixels counted from pixels with smaller brightness values among pixels with brightness values higher than the lower threshold value are pixels of blood vessels. In addition, the blood vessel determination device 18 determines that, among the respective plural pixels configuring the image, the second predetermined number of pixels counted from pixels with higher brightness values among pixels with brightness values lower than the upper threshold value are pixels that are not of blood vessels. Thus, blood vessels appearing in the image may be determined easily. Specifically, because the blood vessels are extracted by a simple algorithm, a reduction in processing load of a microcomputer that is a subject executing the processing may be enabled.

The blood vessel determination device according to this exemplary embodiment acquires plural images captured in a predetermined time interval and generates an averaged image in which the plural images are averaged by averaging brightness values of pixels at corresponding positions contained in the plural images. Based on the brightness values of the plural pixels configuring the averaged image, the blood vessel determination device determines that the first predetermined number of pixels counted from pixels with smaller brightness values are of blood vessels, and determines that the second predetermined number of pixels counted from pixels with higher brightness values are not of blood vessels. The processing of averaging is arithmetic processing with a relatively low computing cost. Images in which blood vessels appear are subject to effects from irregular components such as pulse waves and the like. Accordingly, by averaging plural images, the effects of irregular components such as pulse rates and the like may be moderated, and blood vessel determination accuracy may be improved even while the computing cost is reduced.

Because aggregations of pixels with brightness values not less than an arbitrary lower threshold value are determined to be regions of blood vessels, even when an object other than a finger impinges on an image, pixels in which this object appears may be excluded from determination by the lower threshold value. Therefore, blood vessels may be determined accurately.

Because regions corresponding to an arbitrary number of pixels counted in order of brightness value from smaller brightness values are determined to be blood vessels, irregularities in accuracy of the blood vessel determination due to irregularities in light amounts outputted from a light source may be suppressed.

Because aggregations of pixels with brightness values not more than an arbitrary upper threshold value are determined to be regions that are not blood vessels, even when an object other than a finger on a screen (for example, the light source itself) impinges on an image, pixels in which this object appears may be excluded from determination by the upper threshold value. Therefore, regions that are not blood vessels may be determined accurately.

Because regions corresponding to an arbitrary number of pixels counted from higher brightness values are determined not to be blood vessels, irregularities in accuracy of determining regions that are not blood vessels due to irregularities in light amounts outputted from a light source may be suppressed.

A conversion to a blood sugar level of the owner of a finger F appearing in an image may be calculated based on a difference between an average of brightness values of pixels in a region depicting blood vessels that is extracted by the blood vessel determination device 18 according to the present exemplary embodiment and an average of brightness values in a different region.

An exemplary embodiment of the present disclosure is described above, but the present disclosure is not limited by modes of the exemplary embodiment described above and numerous modifications may be embodied.

For example, in the exemplary embodiment described above, an example is described in which the example of a part of a body is a finger, but this is not limiting. A part of a body that is to be an object of blood vessel determination may be any part.

All publications, patent applications, and technical standards mentioned in this specification are herein incorporated by reference to the same extent as if each individual publication, patent application, or technical standard was specifically and individually indicated to be incorporated by reference.

What is claimed is:

1. A blood vessel determination device, comprising:
a memory; and
a processor coupled to the memory, the processor being configured to:
acquire an image captured while near-infrared light is illuminated at a part of a body, and,
based on brightness values of a plurality of pixels configuring the acquired image, determine that a predetermined number of pixels counted in order of brightness value from a pixel with a lowest brightness value represent blood vessels.

2. The blood vessel determination device according to claim 1, wherein the processor is configured to:
determine that a first predetermined number of pixels counted in order of brightness value from the pixel with the lowest brightness value represent blood vessels, and
determine that a second predetermined number of pixels counted in order of brightness value from a pixel with a highest brightness value do not represent blood vessels.

3. The blood vessel determination device according to claim 2, wherein the processor is further configured to:
arrange the brightness values of the plurality of pixels configuring the acquired image in ascending order, and
based on the arranged brightness values of the plurality of pixels,
determine that the first predetermined number of pixels counted from the pixel with the lowest brightness value represent blood vessels, and
determine that the second predetermined number of pixels counted from the pixel with the highest brightness value do not represent blood vessels.

4. The blood vessel determination device according to claim 1, wherein the part of the body is a finger.

5. The blood vessel determination device according to claim 2, wherein the processor is further configured to:
acquire a plurality of the images, which are captured at a predetermined time interval, by averaging brightness values of pixels at corresponding positions contained in the plurality of images, generate an averaged image in which the plurality of images are averaged,
based on brightness values of a plurality of pixels configuring the averaged image that is generated, determine that the first predetermined number of pixels counted from the pixel with the lowest brightness value represent blood vessels, and
determine that the second predetermined number of pixels counted from the pixel with the highest brightness value do not represent blood vessels.

6. The blood vessel determination device according to claim 1, wherein the processor is further configured to:
determine that, among pixels with higher brightness values than a first threshold value, a first predetermined number of pixels counted from a pixel with a lowest brightness value represent blood vessels; and
determine that, among pixels with lower brightness values than a second threshold value, a second predetermined number of pixels counted from a pixel with a highest brightness value do not represent blood vessels.

7. A blood vessel determination method, comprising, by a computer:
acquiring an image captured while near-infrared light is illuminated at a part of a body; and,
based on brightness values of a plurality of pixels configuring the acquired image, determining that a predetermined number of pixels counted in order of brightness value from a pixel with a lowest brightness value represent blood vessels.

8. A non-transitory storage medium storing a blood vessel determination program that is executable by a computer to perform processing, the processing comprising:
acquiring an image captured while near-infrared light is illuminated at a part of a body; and,
based on brightness values of a plurality of pixels configuring the acquired image, determining that a predetermined number of pixels counted in order of brightness value from a pixel with a lowest brightness value represent blood vessels.
